(19) United States
(12) Patent Application Publication
(10) Pub. No.: US 2025/0127525 A1
(43) Pub. Date: Apr. 24, 2025

(54) INTERNALLY COOLED BONE CUTTING TOOLS

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Darren J. Wilson, Hull (GB); Brett J. Bell, Mendon, UT (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/797,648

(22) Filed: Aug. 8, 2024

Related U.S. Application Data
(60) Provisional application No. 63/544,683, filed on Oct. 18, 2023.

(51) Int. Cl. A61B 17/17; A61B 17/16

(52) U.S. Cl. CPC A61B 17/1764; A61B 17/1642; A61B 2217/007

(57) ABSTRACT

A bone cutting device, such as a burr or saw blade, is irrigated to cool not only the bone cutting device itself, but also the bone of the subject on which a bone resection procedure is being performed. The bone cutting device can have one or more (e.g., a plurality of) internal flow channels that eject a cooling medium (e.g., water or an inert gas) from the bone cutting device. This emission of the cooling medium can not only flush bone chips from the resection site, thereby reducing frictional heating of the bone cutting device, but can also provide internal cooling to the bone cutting device while flowing through the one or more internal flow channels and also can cool the surface of the bone via conductive heat transfer (e.g., by the flow stream of the cooling medium impinging on the bone surface).

### CROSS-REFERENCE TO RELATED APPLICATIONS

This is a non-provisional of, and claims the benefit of the filing date of, pending U.S. provisional patent application No. 63/544,683, filed Oct. 18, 2023, entitled “Internally Cooled Bone Cutting Tools” the entirety of which application is incorporated by reference herein.

### FIELD OF THE DISCLOSURE

The present disclosure relates generally to orthopedic tools and methods for reducing heat in a bone during a resection operation being performed on the bone. Moreover, the present disclosure relates to internally cooled orthopedic tools and associated methods.

### BACKGROUND OF THE DISCLOSURE

During conventional bone resection procedures, such as burring or cutting, localized heating of the bone and cutting tool occurs. It is known that, when the temperature of the bone reaches a high enough temperature, irreversible thermal and mechanical damage may be caused to the bones and soft tissues. Heat generation during bone resection can result from several sources, such as, for example, frictional forces generated between the cutting surface of the cutting tool and the bone being resected and plastic deformation of the bone chips. During rotary bone resection procedures, such as burring, heating of the bone can also be cause due to friction between the bone chips and the cutting flutes of the burr. Furthermore, some of the heat generated within the cutting tool transfers to the resected bone from the internal bearings located in the cutting tool, via conductive heating.

The thermal conductivity coefficient of bone is negligible, ranging generally from about 0.38 Watts per meter Kelvin (W/mK) to about 2.3 W/mK. Thus, due to low bone thermal conductivity, heat remains on the burring site, thus elevation of local temperature occurs, with the nature of bone alkaline phosphatase being subjected to change. This results in thermal necrosis, the death of bone tissue and loss of mechanical strength in the resected zone.

Thermal necrosis depends on two factors: the temperature level and duration of exposure to that temperature. Some researchers have specified a temperature threshold for thermal necrosis, below which no significant impact is exerted on the bone tissue, but above that, the bone cells are irreversibly heat affected. The thermal threshold for necrosis is exposure to a temperature of 47° C. for 1 minute[2]. According to experimental tests conducted on the bone, for each degree increase in the temperature, the tolerable period for maintaining viable bone tissue decreases exponentially. Therefore, when this period is reduced to 30 s at 48° C., until finally at 53° C., the thermal exposure period is reduced to a fraction of a second and thermal necrosis occurs in real time. Burrs typically remain in contact with the tissue longer than a surgical saw blade to create a distal cut during total knee surgery. Moreover, metallic cutting blocks typically help dissipate some of the heat generated from the cutting tool. Consequently, on average, burring produced higher temperatures than sawing during either a partial or total knee replacement surgery, thus necessitating an effective means of cooling.

FIGS. 1A-1C show surface temperatures of bone being resected and also of the cutting device, here, a burr, which is not cooled. FIG. 1A is an image showing surface temperatures of the bone and burr. FIGS. 1B and 1C are graphical plots showing the surface temperature of the burr and of the bone, respectively, from the image of FIG. 1A, which was captured immediately after retraction of the burr from the resection site. The relatively high thermal conductivity of the burr allows the surface of the burr to cool rapidly to about 87.4° C. However, due to the lower thermal conductivity of the bone, the temperature of the bone at the resection site is about 96.4° C., which is well above the critical threshold for the onset of osteonecrosis. It should be noted that, since the image and accompanying data shown in FIGS. 1A-1C were obtained after retraction of the burr from the resection site, the temperatures of the burr and of the bone would be even higher than the temperatures noted herein during live surgery.

FIGS. 12A and 12B show surface temperatures of bone being resected and also of the cutting device, here, an oscillating tip saw blade, which is not cooled. FIG. 12A is an image showing surface temperatures of the bone and saw blade. FIG. 12B is a graphical plot showing the surface temperature of the saw blade as a function of time during a bone resection procedure. As shown, a maximum saw blade surface temperature of about 133° C. was recorded during bone resection.

Furthermore, since the image and data shown in FIGS. 1A-1C and 12A-12B were obtained during a bone resection procedure on a cadaver, the generally higher temperature of bone within a living subject may cause the temperature of the bone and cutting device to be even higher than the temperatures shown in FIGS. 1A-1C and 12A-12B, although the flow of blood in a living subject may counteract this effect to at least some degree, if not entirely.

Adequate fixation of an implant device to/within the host bone is a critical factor in the success of resurfacing a joint, especially in the case of cementless fixation. Hence, the use of a conventional, non-cooled burr or sawblade may contribute to resorption at the bone implant surface, which could account for early radiographic lucencies and potential loosening of the implant components.

### BRIEF DESCRIPTION OF THE DRAWINGS

By way of example, specific examples of the disclosed device will now be described, with reference to the accompanying drawings, in which:

FIG. 1A is a thermal imaging photograph showing temperatures of a conventional burr and a bone immediately after a bone resection procedure has been performed using a conventional burr;

FIGS. 1B and 1C are plots of temperatures of the burr and bone surface in the thermal imaging photograph of FIG. 1A;

FIG. 2 shows a handpiece with an example irrigated burr assembly attached to the handpiece in accordance with one or more features of the present disclosure;

FIGS. 3-6 are various views to show features of the example irrigated burr assembly shown in FIG. 2;

FIGS. 7-9 are various views of an alternate example of an irrigated burr assembly in accordance with one or more features of the present disclosure, the irrigated burr assembly suitable for attachment to and use with the handpiece shown in FIG. 2;

FIG. 10 is a schematic illustration of a handpiece for a tool configured to supply a cooling medium in the form of CO₂ axially through a burr with a flow channel formed internal to and along the length of the burr;

FIG. 11 schematically shows effects in temperature reduction from water cooling and CO₂ cooling compared to a conventional burr during a bone resection procedure;

FIG. 12A is a thermal imaging photograph showing temperatures of a conventional oscillating bone saw and a bone during a bone resection procedure;

FIG. 12B is a plot of temperature of the saw blade in the thermal imaging photograph of FIG. 12A;

FIGS. 13A and 13B shows various examples of a cutting saw blade with internal flow channels formed therein in accordance with one or more features of the present disclosure, FIG. 13A including longitudinal flow channels, FIG. 13B including longitudinal and transverse flow channels;

FIG. 14 is an alternate view of the oscillating saw blade shown in FIG. 13A, the oscillating saw blade including flow channels formed internal to the body of the saw blade;

FIG. 15 is an internal view of an alternate example of an irrigated saw blade having flow channels formed internal thereto in accordance with one or more features of the present disclosure;

FIG. 16A is an internal view of another example of an irrigated saw blade having flow channels formed internal thereto in accordance with one or more features of the present disclosure;

FIG. 16B is an exploded view of an example inlet for the irrigated saw blade shown in FIG. 16A; and

FIGS. 17-21C are various views of another example irrigated saw blade having flow channels formed internal thereto in accordance with one or more features of the present disclosure.

FIG. 22A is a view of a cutting end of another example irrigated saw blade having one flow channel formed internal thereto in accordance with one or more features of the present disclosure.

FIG. 22B is a view of a cutting end of another example irrigated saw blade having three flow channels formed internal thereto in accordance with one or more features of the present disclosure.

FIG. 22C is a view of a Luer inlet laser welded to an example irrigated saw blade with flow channel(s) formed internal thereto, in accordance with one or more features of the present disclosure.

FIGS. 23A and 23B are temporal graphical plots of bone surface temperature using a conventional saw blade and an internally irrigated saw blade, respectively, in accordance with one or more features of the present disclosure.

FIG. 24A shows example irrigated saw blades with internal flow channels that can be formed by additive manufacturing for internally cooling the sawblade.

FIGS. 24B and 24C are photographs of saw blades having internal flow channels formed therein, the saw blades shown having been formed by additive manufacturing.

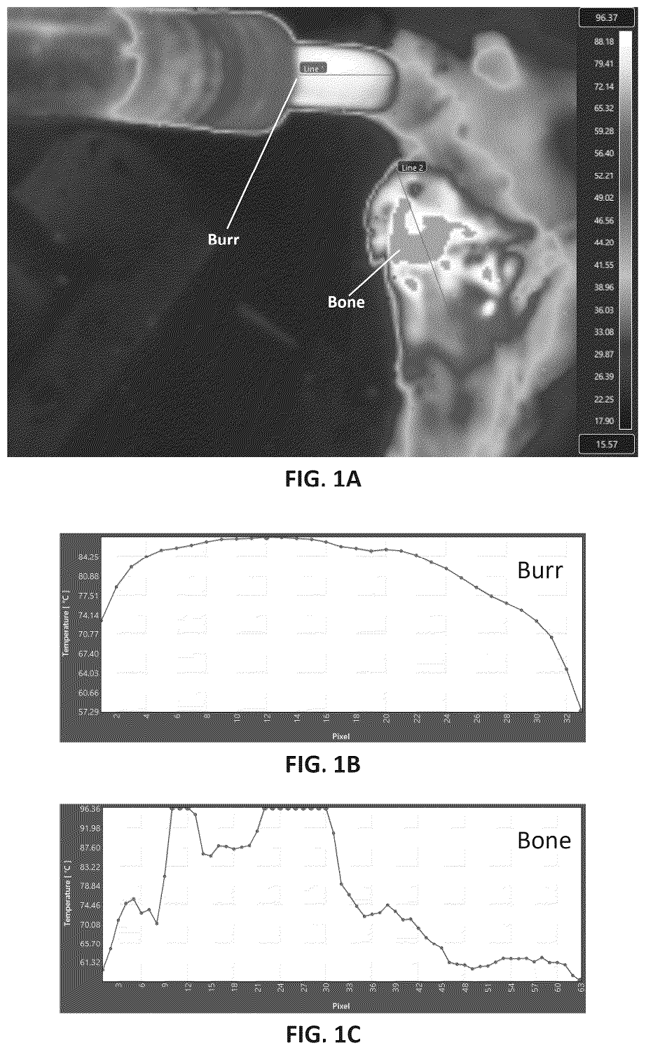

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

Various features or the like of a bone cutting device will now be described more fully herein with reference to the accompanying drawings, in which one or more features of an at least partially internally cooled cutting device for use during bone resection procedures is/are disclosed. It should be appreciated that the various features may be used independently of, or in combination, with each other. It will be appreciated that the various example cutting devices disclosed herein may be embodied in many different forms and may selectively include one or more concepts, features, or functions described herein. As such, the presently disclosed cutting devices should not be construed as being limited to the specific examples set forth herein. Rather, these examples are provided so that this disclosure will convey certain features of such cooled cutting devices to those skilled in the art.

In accordance with one or more features of the present disclosure, examples of various cutting devices, such as saw blades, burrs, and the like, which are used, for example, during bone resection procedures to reduce temperatures of the cutting device and also of the bone being resected. These cutting devices are cooled during operation by a cooling medium, examples of which include water, a saline solution, carbon dioxide (CO₂), and nitrogen (N₂).

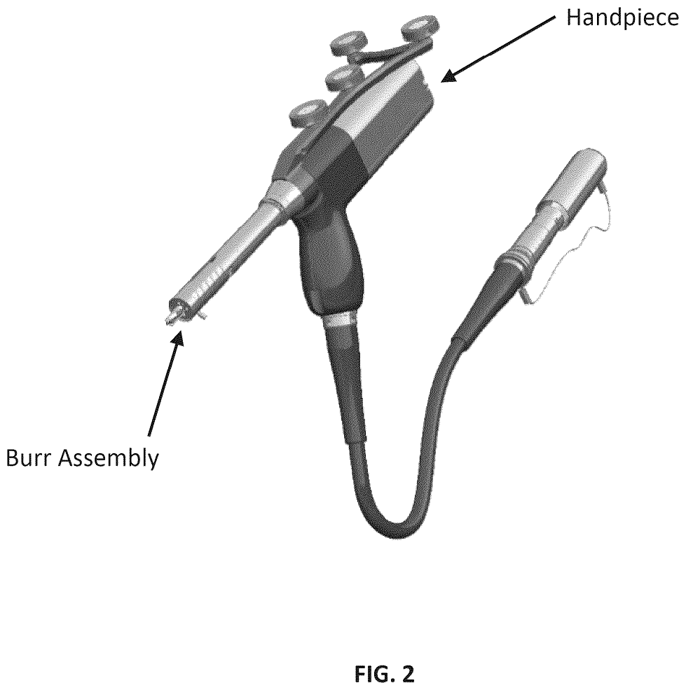

FIG. 2 shows an example handpiece for use by a surgeon in performing a burring technique for bone resection, the handpiece having a burr assembly operably attached thereto. The burr assembly shown in FIG. 2 is cooled by provision of a cooling medium, such as water, a saline solution, CO₂, and N₂.

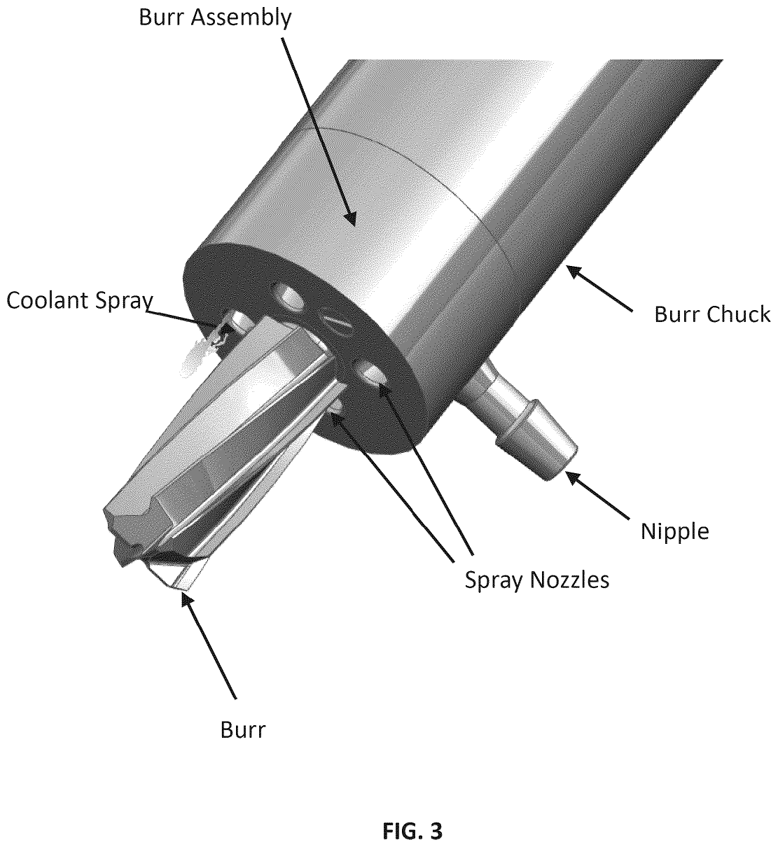

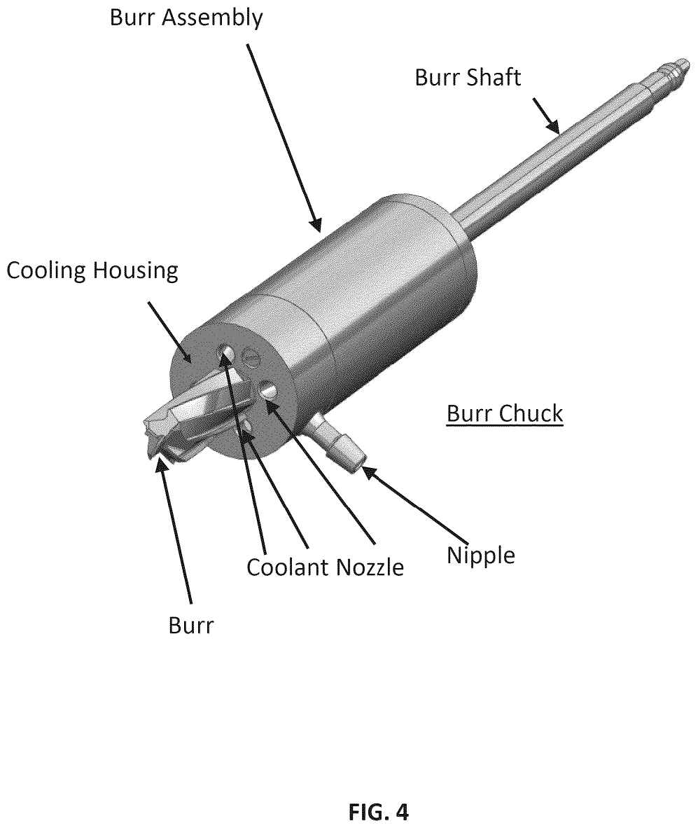

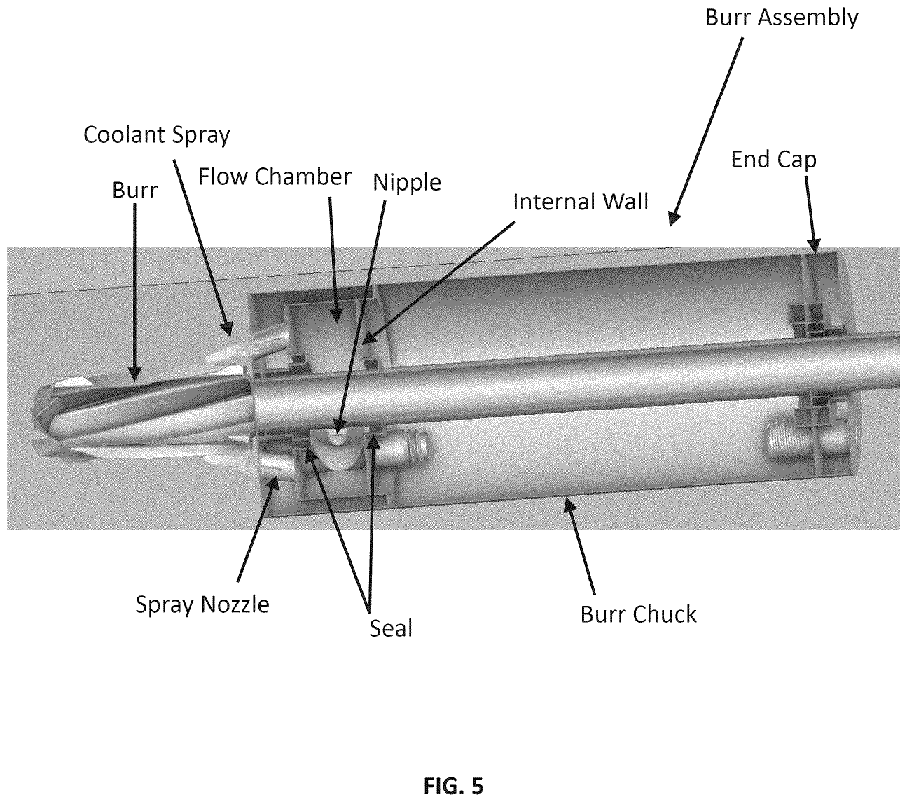

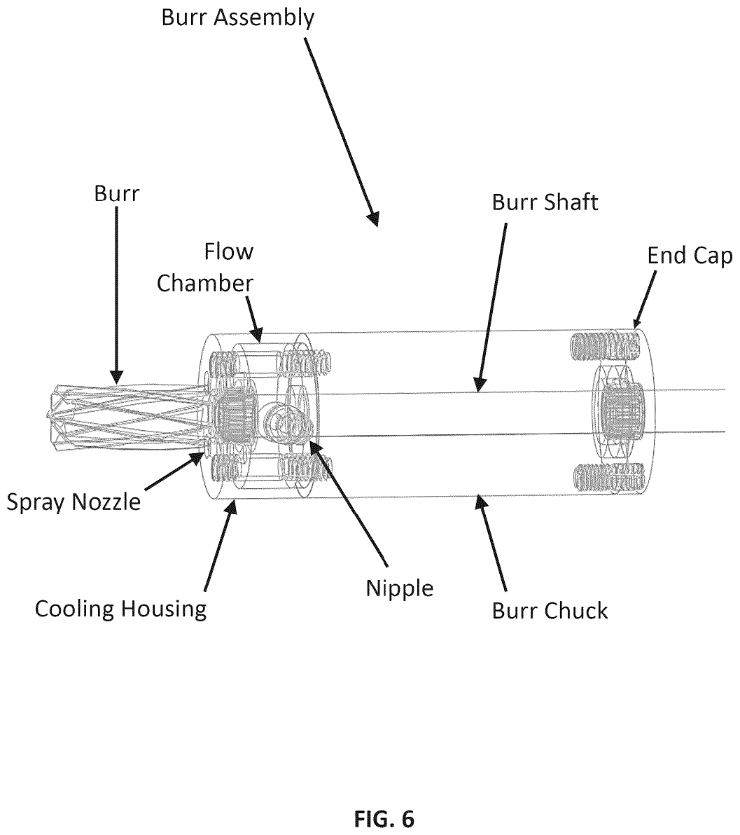

FIGS. 3-6 show several views for an example burr assembly suitable for use with a conventional handpiece, such as the handpiece shown in FIG. 2. The example burr assembly shown in FIGS. 3-6 is internally cooled by a flow of a cooling medium through at least a portion of the burr assembly. The burr assembly includes a burr shaft with a burr at the distal end thereof. The proximal end of the burr shaft is operably attached to the handpiece, such that the handpiece drives a rotation of the burr shaft and, thus necessarily, of the burr itself for bone resection. A burr chuck concentrically surrounds the burr shaft and is spaced apart from the burr shaft by, for example, needle bearings or any other suitable type of rotary support device. The needle bearings are supported in an end cap and in a cooling housing to space the burr chuck radially apart from the burr shaft during operation.

The end cap is affixed on (e.g., by screws passing through the end cap and into the burr chuck) a first end of the burr chuck. A cooling housing is affixed on (e.g., by screws passing through the cooling housing and into the burr chuck) a second end of the burr chuck. The first end of the burr chuck is closest to the handpiece and the second end of the burr chuck is closest to the burr. The first and second ends of the burr chuck may be referred to as opposite longitudinal ends of the burr chuck from each other. Thus, the burr chuck is radially spaced apart from the outer circumference of the burr shaft. The cooling housing includes an internal flow chamber, which is supplied with a cooling medium via a nipple, or other suitable inlet. The cooling medium enters the flow chamber through the nipple and is then ejected under hydraulic pressure within the flow chamber from a plurality of spray nozzles. The hydraulic pressure is, in some instances, the same as the supply pressure at which the cooling medium is supplied at the nipple.

FIG. 3 schematically shows a coolant spray being emitted from each of the spray nozzles. While the spray nozzles may extend only in the longitudinal direction, it is advantageous for the spray nozzles to be inclined radially inwardly, towards the burr, at least to some degree, so that the coolant spray impinges upon the burr and/or a bone resection site axially beyond a distal tip of the burr. Thus, the cooling medium is advantageously used to assist in bone fragment removal from the cutting flutes of the burr to prevent “clogging” of such cutting flutes that would otherwise result in a reduction in cutting efficiency. As shown clearly in FIG. 5, the direction of extension of the spray nozzles has both a longitudinal component and a radial component, as defined by, for example, a polar coordinate system. In some instances, the direction of extension of the spray nozzles may also have a circumferential component, such as would induce a swirling pattern. The circumferential component can be in the same or reverse direction of the cutting surfaces of the burr and/or of the direction of rotation of the burr during use. In the example shown, the burr chuck includes a quantity of four (4) spray nozzles and thus, during operation, there are correspondingly a quantity of four (4) coolant sprays directed towards the burr for cooling. Any suitable quantity of spray nozzles may be formed in the distal end of the cooling housing and the quantity of coolant sprays emitted from the spray nozzles corresponds to (e.g., is the same as, or a multiple of) the quantity of the spray nozzles.

The operation of the burr and the emission of coolant spray from the spray nozzles can be independent of each other, meaning that the rotation of the burr is caused by rotation of the burr shaft by the handpiece, while the emission of coolant spray from the spray nozzles is controlled by the supply pressure of the coolant medium into the flow chamber at the nipple from a coolant supply source, such as a coolant medium reservoir. Thus, the burr shaft can spin the burr without coolant spray being emitted from the spray nozzles and, conversely, coolant spray can be emitted from the spray nozzles without the burr spinning.

Seals are provided within the cooling housing to prevent escape of the cooling medium from the flow chamber during use, except through the spray nozzles. The seals press against the outer surface of the burr shaft to prevent such leakage.

The burr chuck sub-assembly components consist of a metal shell case, which can be split into two parts for ease of assembly, two metal end closures, two washers/O-rings, two needle roller bearings, inner and outer bushings and four M2 screws for mechanical and hermetic sealing. The burr and open-circuit chuck work together as a combined tool having a cooling system that spans from the outer sleeve to the cutting flutes on the burr, which witnesses the greatest amount of frictional heat. The cooling chuck can accommodate different size burr heads, e.g., 5, 6 and, 7 mm diameter and designs, e.g., spherical and cylindrical. With regards to the cooling system, the coolant comes from the reservoir and circulates within the open-circuit design exiting from the divergent holes located at the distal end of the chuck onto the flutes of the burr.

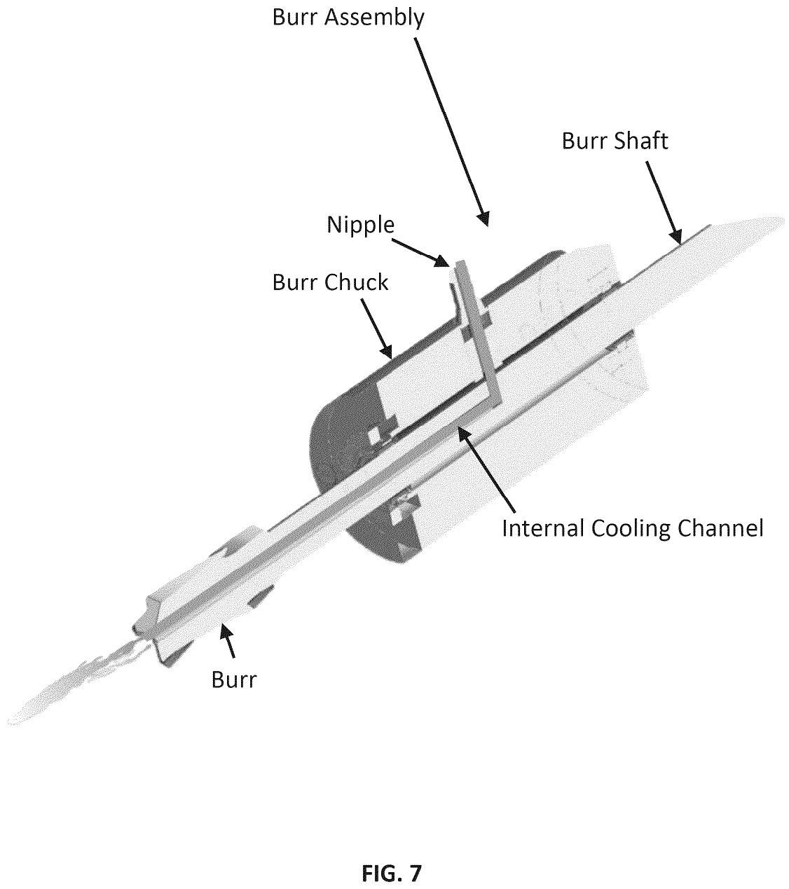

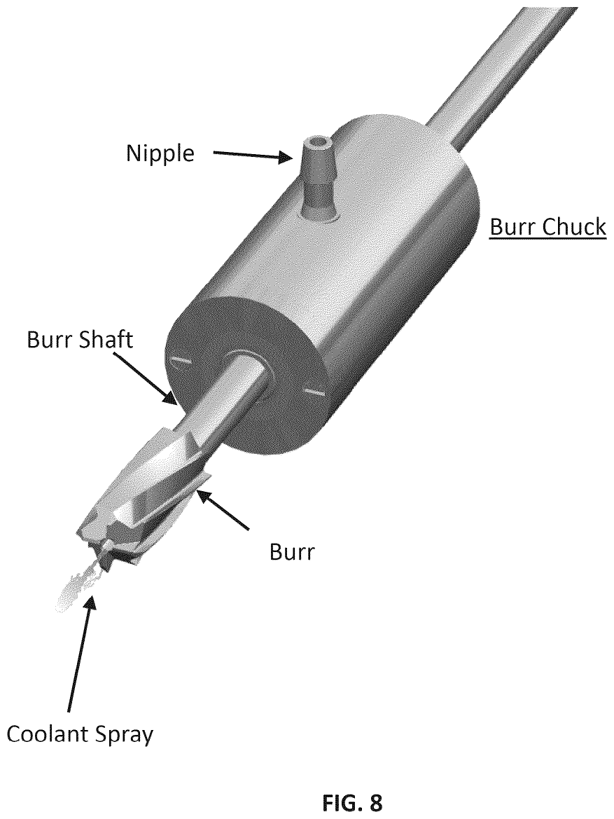

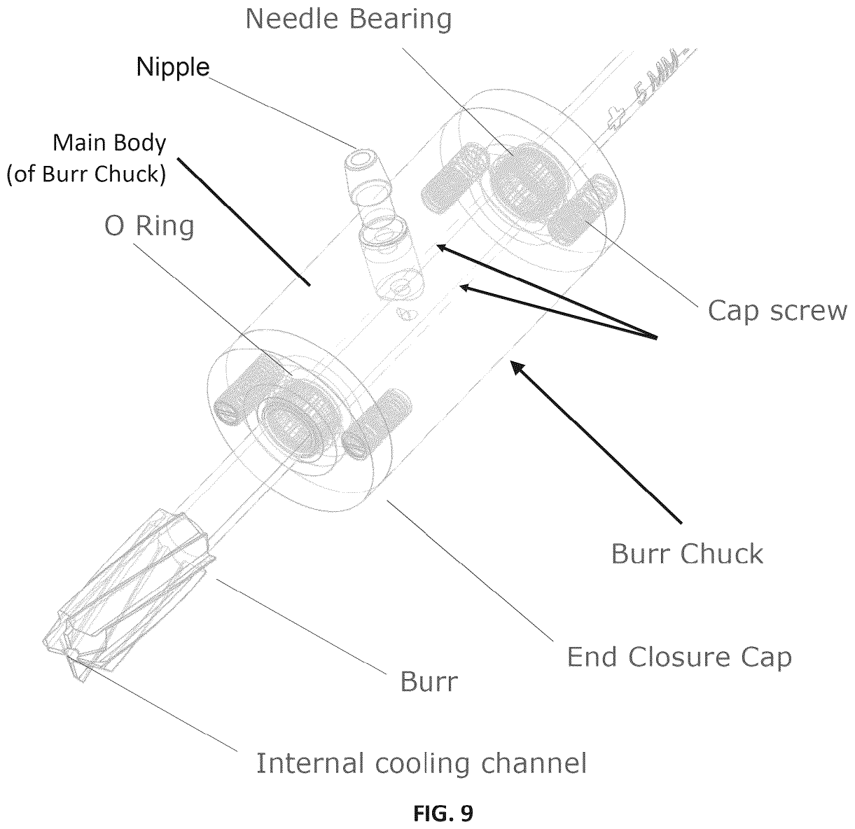

FIGS. 7-9 show several views for another example burr assembly suitable for use with a conventional handpiece, such as the handpiece shown in FIG. 2. Unlike the externally cooled, impinging cooling style of the burr assembly shown in FIGS. 3-6, the burr assembly shown in FIGS. 7-9 has flow path for the cooling medium that is entirely internal to the burr assembly. Similar to the burr assembly of FIGS. 3-6, the burr assembly of FIGS. 7-9 has a burr chuck that is provided annularly around an outer circumference of a burr shaft, such that the burr shaft extends axially through the burr chuck. An end cap is attached to each of the opposing longitudinal ends of the burr chuck in the same manner as is described in the example shown in FIGS. 3-6.

The burr assembly of FIGS. 7-9 has a nipple, which has an axially-extending flow channel therethrough, which is attached to an outer surface of and extends into and through the main housing of the burr chuck. The main housing is the portion of the burr chuck that is between the end caps and to which the end caps are attached. The flow channel extends through the nipple in a radially inward direction, such that an outlet of the flow channel is against the outer circumferential surface of the burr shaft, internal to the burr chuck.

The burr shaft has formed therein an inlet passage that extends from the outer circumferential surface of the burr shaft to intersect with a longitudinally-extending internal cooling channel that extends from the inlet passage of the burr shaft to the distal end of the burr, so as to allow a flow of the cooling medium through the nipple, into the inlet passage, and through the internal cooling channel, such that the cooling medium is ejected as a stream or spray from the distal end, or tip, of the burr. The inlet passage and the internal cooling channel are inclined at a non-zero angle (e.g., perpendicular) relative to each other.

There is a flow chamber defined between the inner circumferential surface of the burr chuck and the outer circumferential surface of the burr shaft. The longitudinal ends of the flow chamber are defined by a seal (e.g., an O-ring) that is secured in place by the respective opposing end caps. Thus, the flow chamber can have a shape of a hollow cylinder. Bearings are provided to maintain a substantially constant radial gap between the burr chuck and the burr shaft. The cooling medium enters the flow chamber through the nipple. Because there is an annular gap between the outer circumferential surface of the burr shaft and the inner circumferential surface of the main body of the burr chuck, the cooling medium enters the flow chamber and can then pass into the inlet passage and through the internal cooling channel of the burr shaft continuously while the burr shaft rotates. Furthermore, because the internal cooling channel extends substantially the entire length of the main body of the burr assembly, such that there is essentially a body of the cooling medium within the flow chamber to act as an intermediate reservoir, the precise location of the inlet passage is not critical, as long as the burr shaft can be secured such that the inlet passage remains internal to the flow chamber, so that the flow of the cooling medium is not interrupted.

The cooling medium enters the flow chamber through the nipple and is then ejected under hydraulic pressure within the flow chamber from the distal end of the internal cooling channel, at the furthest tip of the burr. The internal cooling channel is formed such that the cooling medium is ejected substantially longitudinally from the internal cooling channel. The hydraulic pressure is, in some instances, the same as the supply pressure at which the cooling medium is supplied at the nipple. Thus, according to this example of the burr assembly, the burr is cooled locally at the tip thereof during cutting, while any bone fragments that could become lodged between the flutes are simultaneously removed and/or washed away by the irrigating flow of the coolant medium.

The operation of the burr and the emission of coolant spray from the internal cooling channel can be independent of each other, meaning that the rotation of the burr is caused by rotation of the burr shaft by the handpiece, while the emission of coolant spray from the internal cooling channel is controlled by the supply pressure of the coolant medium into the flow chamber at the nipple from a coolant supply source, such as a coolant medium reservoir. Thus, the burr shaft can spin the burr without coolant spray being emitted from the internal cooling channel and, conversely, coolant spray can be emitted from the internal cooling channel without the burr spinning.

The example burr assemblies disclosed in FIGS. 3-6 and in FIGS. 7-9 both advantageously act to irrigate the resection site to flush out bone chips and other debris generated during bone resection, which has been found to account for about 60-70% of total heat generation during bone resection. The main cutting action in these example burr assemblies is along the longitudinal axis of the tool at the cutting flutes, or surfaces, of the burr, which generates the greatest amount of friction-induced heat. These example burr assemblies both utilize so-called open-circuit cooling systems that spray a cooling medium from a cooling source (e.g., a reservoir) to irrigate the bone resection site. The term open-circuit is used to specify that the cooling medium is not reused or recirculated to cool the bone resection site after is it emitted from either of the burr assemblies. The burr chucks of the burr assemblies disclosed herein are both re-usable and can be mounted onto any of a plurality of differently-sized burrs. The burr chuck connected to the coolant tubing (i.e., at the nipple) is held in a stationary position, while the burr shaft and burr rotate internal to the burr chuck. The bearings disclosed herein allow the burr shaft and the burr chuck to rotated freely relative to each other.

In any of the example cutting devices disclosed herein, the cooling medium can be, for example, an inert gas, such as CO₂ or N₂, which can be used to lubricate and cool the burr simultaneously. Unlike when the cooling medium is in the form of liquid water, obstruction of the cutting flutes can still occur to at least some degree by the formation of a paste-like material from bone chip accumulation and mixing with the water-based cooling medium, this paste preventing the water from reaching deeper within a bone resection site where the end of the burr is located; this is especially true for the example burr assembly disclosed in FIGS. 3-6, whereas the flow of the cooling medium from the tip of the burr in the example burr assembly disclosed in FIGS. 7-9 originates from within the burr within the bone resection site. When using an inert gas as the cooling medium there is no bone chip accumulation within the bone resection site that would otherwise result in an increase in friction and, thus, heat. An inert gas is also associated with a reduced risk of infection compared to water-based cooling mediums while, especially in the example burr assembly of FIGS. 3-6, allowing for enhanced cooling at greater depths within the bone resection site.

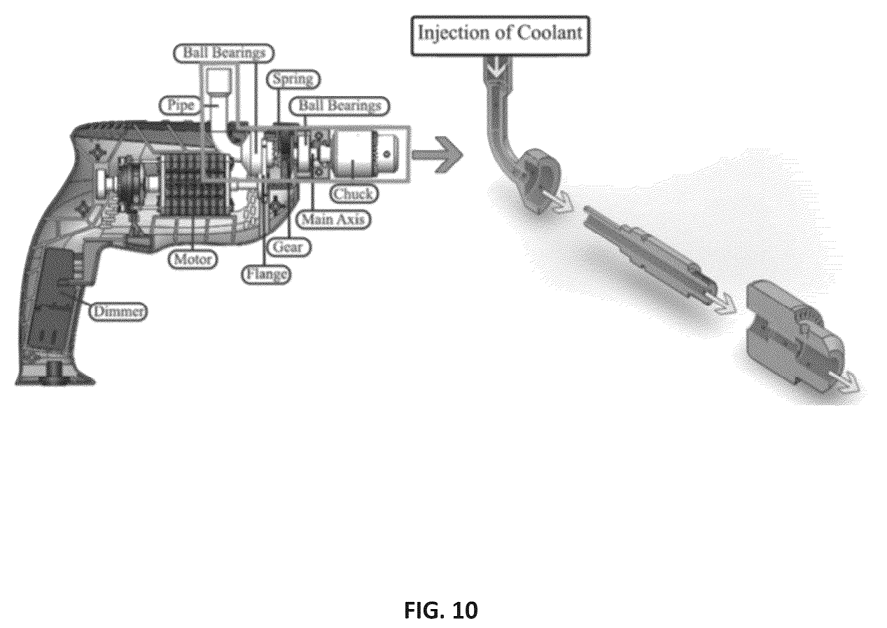

FIG. 10 is a schematic illustration of a handheld device that is suitable for use with an inert gas for the cooling medium.

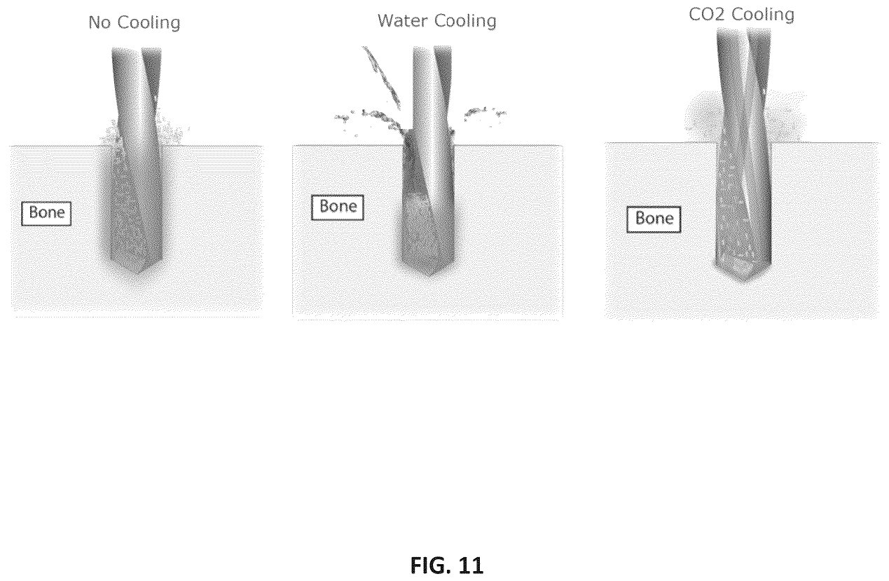

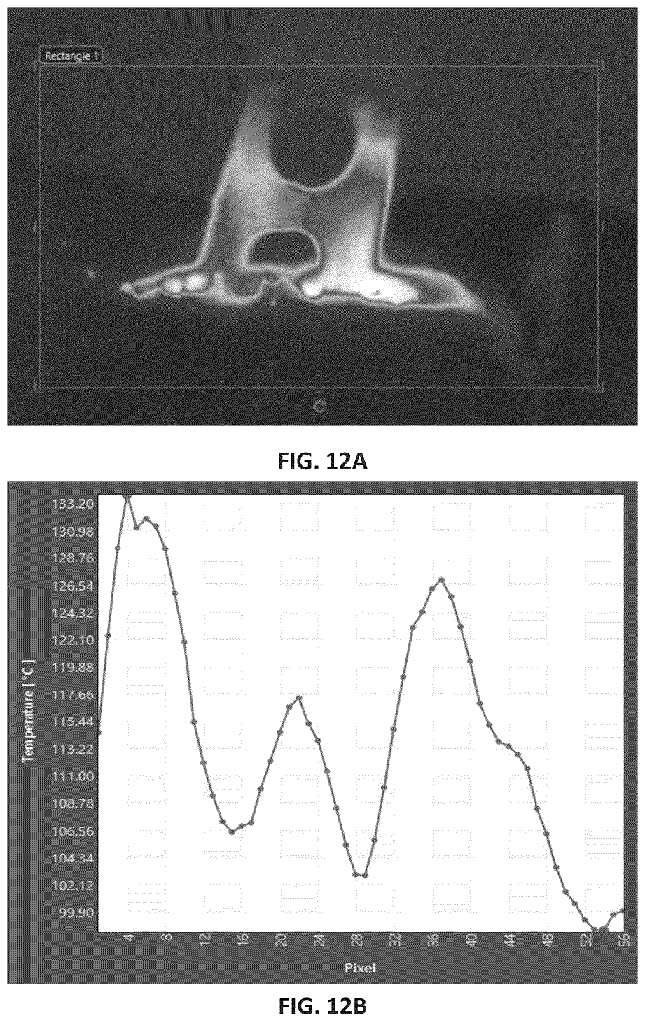

FIG. 11 is a schematic illustration showing bone chip accumulation for a burr with no cooling (left image), a water-based cooling medium (center image), and inert gas-based cooling medium (right image). As shown in FIG. 11, bone chip accumulation occurs with water cooling, which obstructs the cutting flutes with paste that prevents the water-based cooling medium from reaching the deeper cutting site. However, as also shown in the right image of FIG. 11, with a cooling medium using an inert gas there is no bone chip accumulation that can otherwise result in an increase in friction.

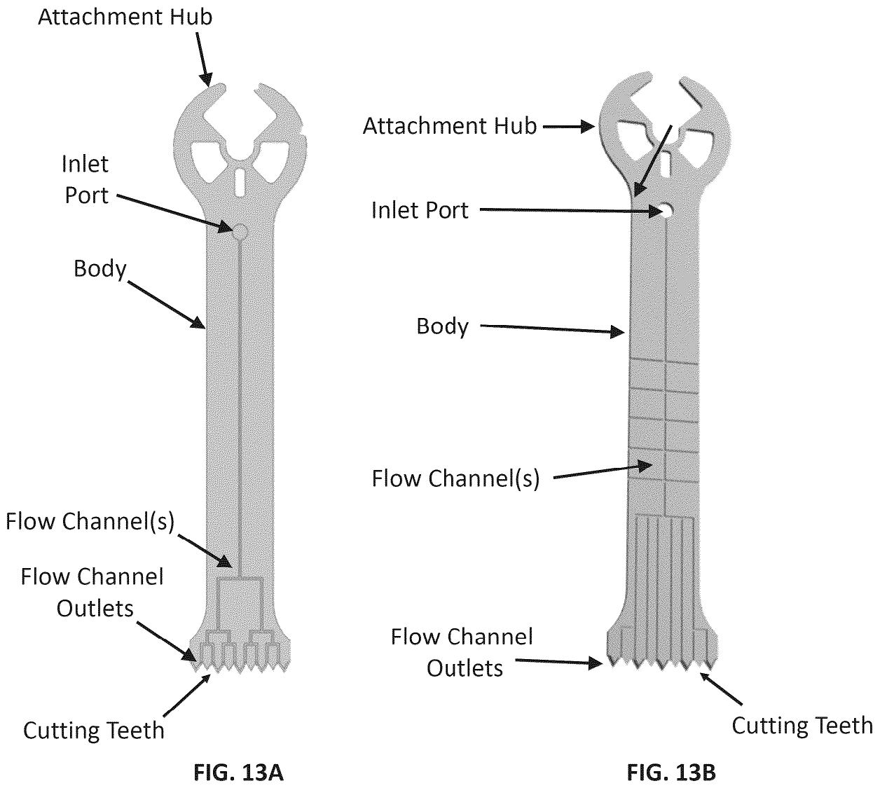

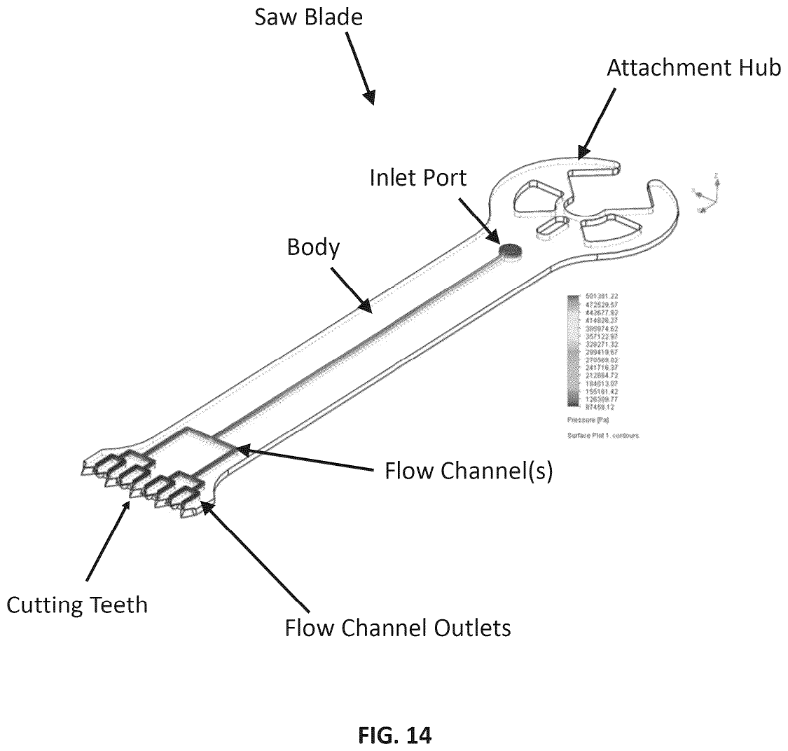

FIGS. 13A, 13B, and 14 show an example oscillating saw blade having a body, an attachment hub formed on a first end of the body, and cutting teeth formed on a second end of the body, opposite the first end. The attachment hub is used for operatively connecting the saw blade to a cutting tool, such as an oscillating cutting tool. The cutting teeth are formed to cut through bone. The saw blade is, for example, from about 0.7 mm to about 1.3 mm thick. The saw blade has, formed internal to the body, a plurality of flow channels that extend from an inlet port (e.g., a hole) to a series of openings that define flow channel outlets at the cutting teeth. These flow channel openings can be formed on the face of the kerf of such an oscillating cutting blade as is shown herein. These internal flow channels can be formed by an internalized CNC process or spark erosion, such that the internal flow channels are in the form of micro-channels. These micro-channels provide a flow of a cooling medium from the inlet port to the flow channel outlets for irrigating the cooling medium into the bone-tool interface. It has been found that the ability to deliver the cooling medium at the bone-tool interface results in a significant reduction of tool wear, cutting force, and specific cutting energy, while also achieving an improved chip fragmentation and microstructure at the bone resection site.

The flow channels shown in FIGS. 13A and 13B can be integrated into a standard mechanical resection saw blade, such as, for example, via spark erosion or any other suitable method of forming such flow channels. The example saw blade shown in FIG. 13A includes longitudinal flow channels (e.g., approximately 0.5 mm wide×0.5 mm deep) that are machined into a standard oscillating saw blade, which can be made out of 304 stainless steel, for example. The example saw blade shown in FIG. 13B includes longitudinal and transverse flow channels (e.g., approximately 0.5 mm wide×0.5 mm deep) that are machined into a standard oscillating saw blade, which can be made out of 304 stainless steel, for example. In both examples of the saw blade, the cooling medium is introduced into the flow channels from the inlet port using, for example, a Luer Fitting, which can be located at least 70 mm proximal from the kerf to allow the saw blade to be inserted freely into a cutting guide without damaging the Luer fitting. This cooling medium is then directed through the internal longitudinal flow channels towards the cutting teeth to both cool the blade and bone in the cutting area of the bone resection site, while also acting to clear the bone chips from the kerf during bone resection.

In some instances, both of the examples shown in FIGS. 13A and 13B may be formed such that the flow channels are entirely internal to (e.g., excluding the inlet(s) and outlet(s) thereof) the saw blade. In some instances, both of the examples shown in FIGS. 13A and 13B may be formed such that the flow channels are formed in an external surface of the saw blade, in which case the saw blade may be mechanically coupled with a substantially identical saw blade (e.g., including a mirrored saw blade) to form a dual-bladed oscillating cutting saw assembly. In an example of such a dual-bladed oscillating cutting saw assembly, the first saw blade is arranged such that the external surface thereof, in which the flow channels thereof are formed, is against (e.g., adjacent to, facing, in a direct manner, etc.) the external surface of a second saw blade, the flow channels thereof being formed in this external surface of the second saw blade that is against the first saw blade.

In the example shown in FIG. 13B, in which the saw blade has both longitudinal flow channels and transverse flow channels, the water outlets for the transverse flow channels, located on the side and kerf of the saw blade, support a low volume high-pressure flow rate, reducing the risk of heat osteonecrosis during a bone resection procedure using such a saw blade.

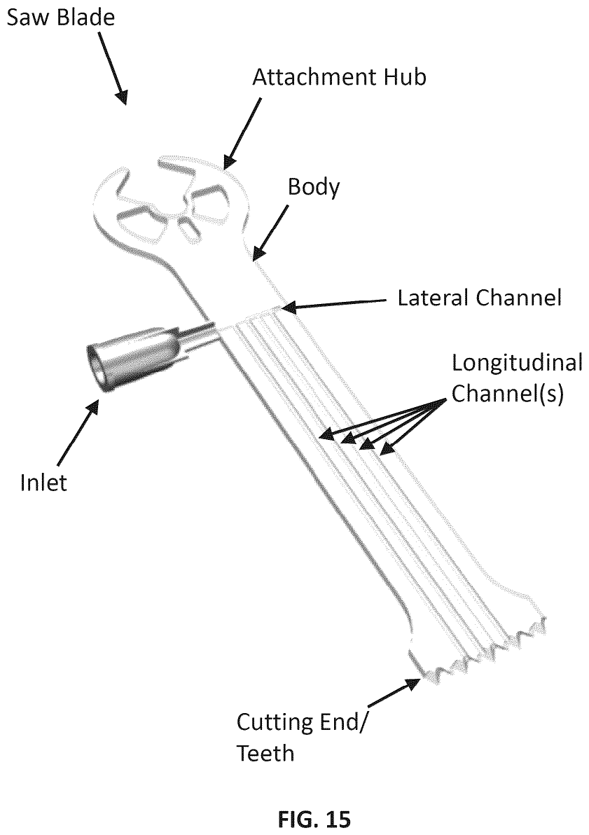

FIG. 15 shows an alternate example of a cutting saw with a laterally-attached inlet that is attached to a 1.35 mm thick body of the saw blade using laser welding, into a lateral channel approximately 0.7 mm in diameter that is internal to the body of the saw blade. The saw blade may also include a plurality of longitudinal channels that are fluidically connected to the lateral channel and extend to the cutting end/teeth of the saw blade to allow for a flow of the cutting medium from the inlet to the flow channel outlets at the cutting end/teeth.

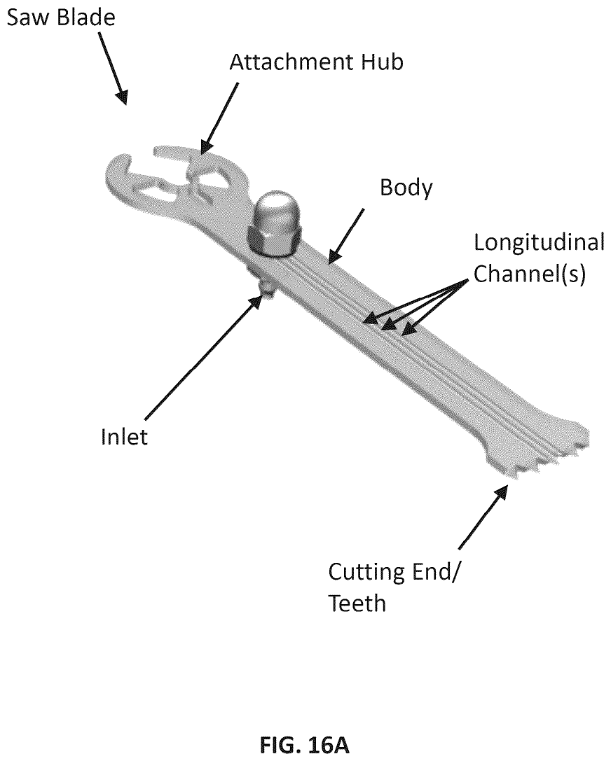

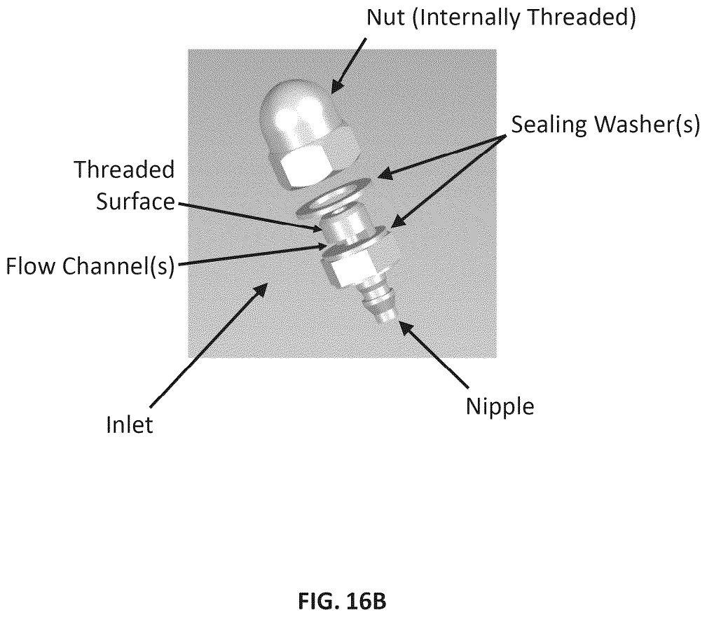

FIG. 16A shows an alternate example of a cutting saw with an inlet that is threadably-secured to, or attached to, the body of the saw blade. The saw blade may also include a plurality of longitudinal channels that are fluidically connected to the inlet and extend to the cutting end/teeth of the saw blade to allow for a flow of the cutting medium from the inlet to the flow channel outlets at the cutting end/teeth.

An exploded view of the example inlet is shown in FIG. 16B. As shown, the inlet includes a nipple that has, on an opposite end thereof, a threaded surface. The threaded surface extends through an inlet port (see, e.g., FIGS. 13A, 13B, and 14) and the inlet is secured to the body of the saw blade by an internally threaded nut being threadably engaged with the threaded surface of the nipple. Sealing washers, or other suitable sealing members, are provided on opposite sides of the body, between the nut and the body and between the nipple and the body. The nipple has, in a portion thereof that is secured to be entirely internal to the body of the saw blade, flow channels that allow a flow of a cooling medium into the flow channels formed in the body of the saw blade. The flow direction of the cooling medium through the nipple (i.e., before exiting the flow channels formed therein) is inclined at a non-zero angle (e.g., perpendicular to) the flow direction of the cooling medium through the flow channels formed in the saw blade. The inlet shown in FIG. 16B is only an example and any suitable type of inlet may be used to introduce a flow of a cooling medium into the flow channels of the saw blade in a substantially or entirely leak-free manner.

The saw blades in any of the examples disclosed herein can be used as double oscillating saw blades to increase stability wit less tendency for bending (“skiving”) during bone resection and deflection during navigation. In such double oscillating saw blades, the flow channels may be formed on an external surface of the individual saw blades. In single-blade saw blades, the flow channels are fully internal to the saw blades between the inlet and the flow channel outlets. Thus, an optimally flat surface, providing a closer contact between bone and prosthesis may encourage better osseointegration.

The saw blades disclosed herein may be formed using an additive manufacturing process.

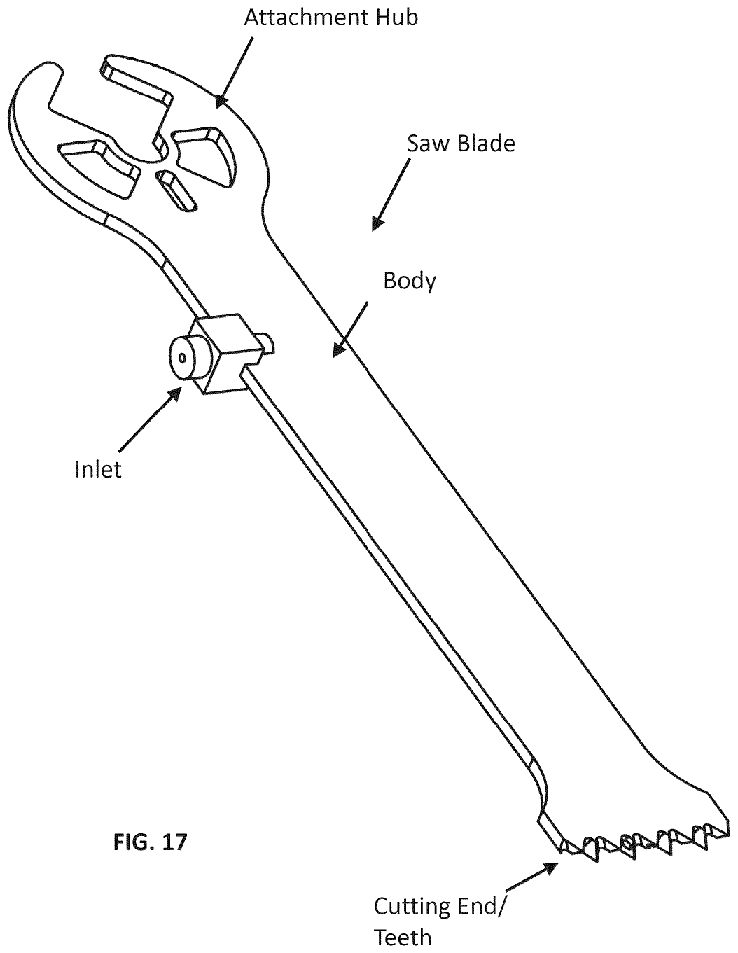

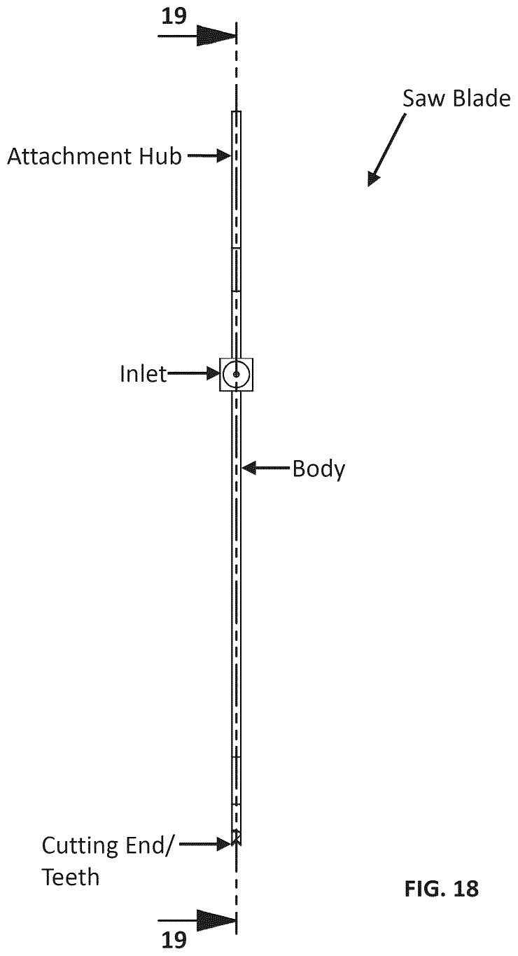

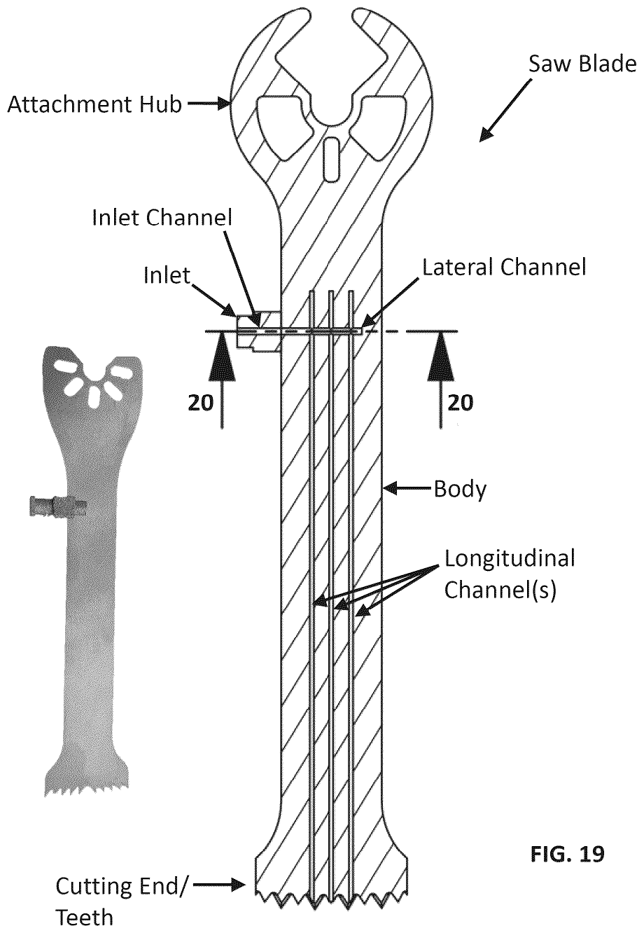

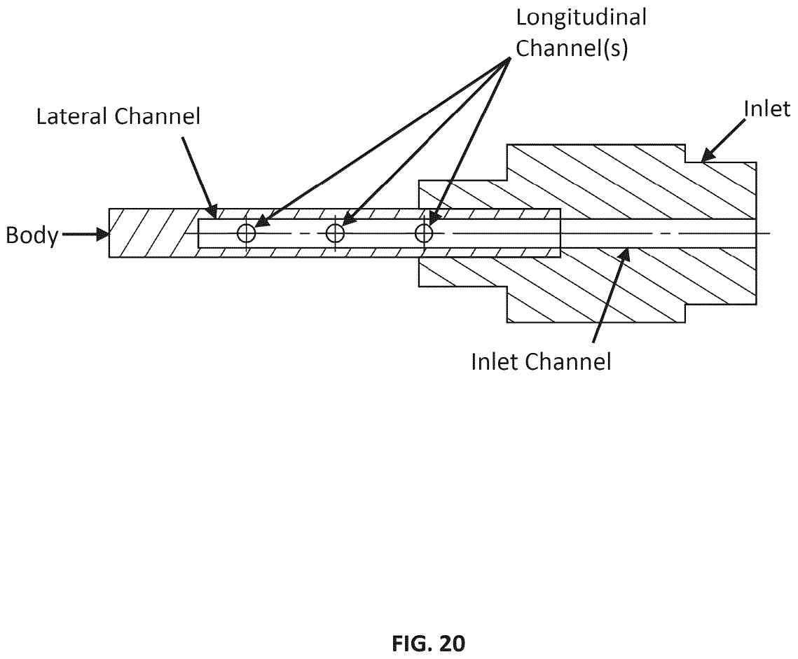

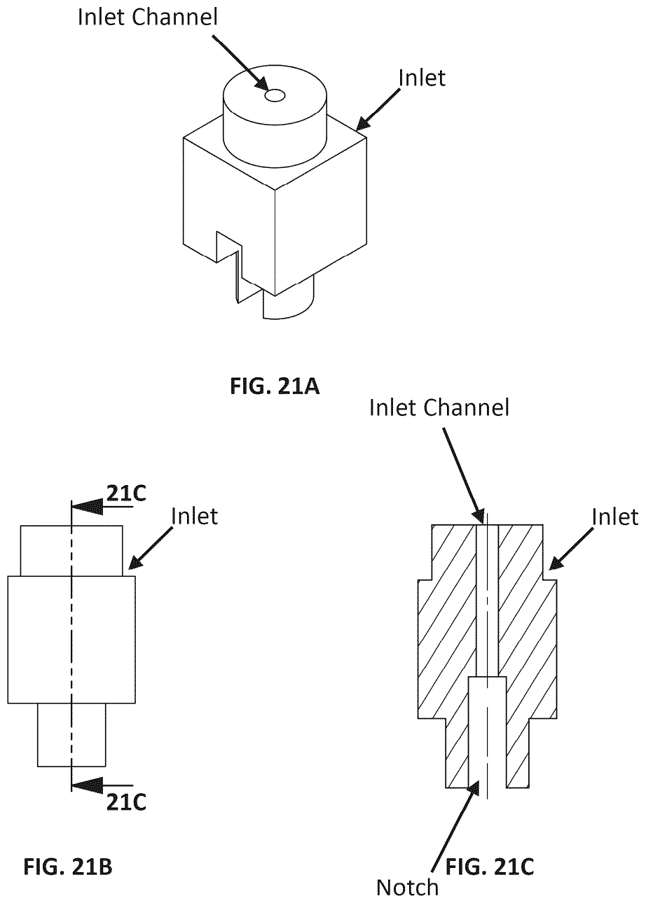

FIGS. 17-21C shows features of another example cutting blade. The cutting blade has a laterally-attached inlet that is attached to the body of the saw blade, into a lateral channel that is internal to the body of the saw blade. The saw blade may also include a plurality of longitudinal channels that are fluidically connected to the lateral channel and extend to the cutting end/teeth of the saw blade to allow for a flow of the cutting medium from the inlet to the flow channel outlets at the cutting end/teeth. FIGS. 21A-21C show features of an inlet, with a notch formed therein that allows for the insertion of the body of the saw blade therein, as well as the inlet channel that allows for a flow of the cooling medium into the lateral channel.

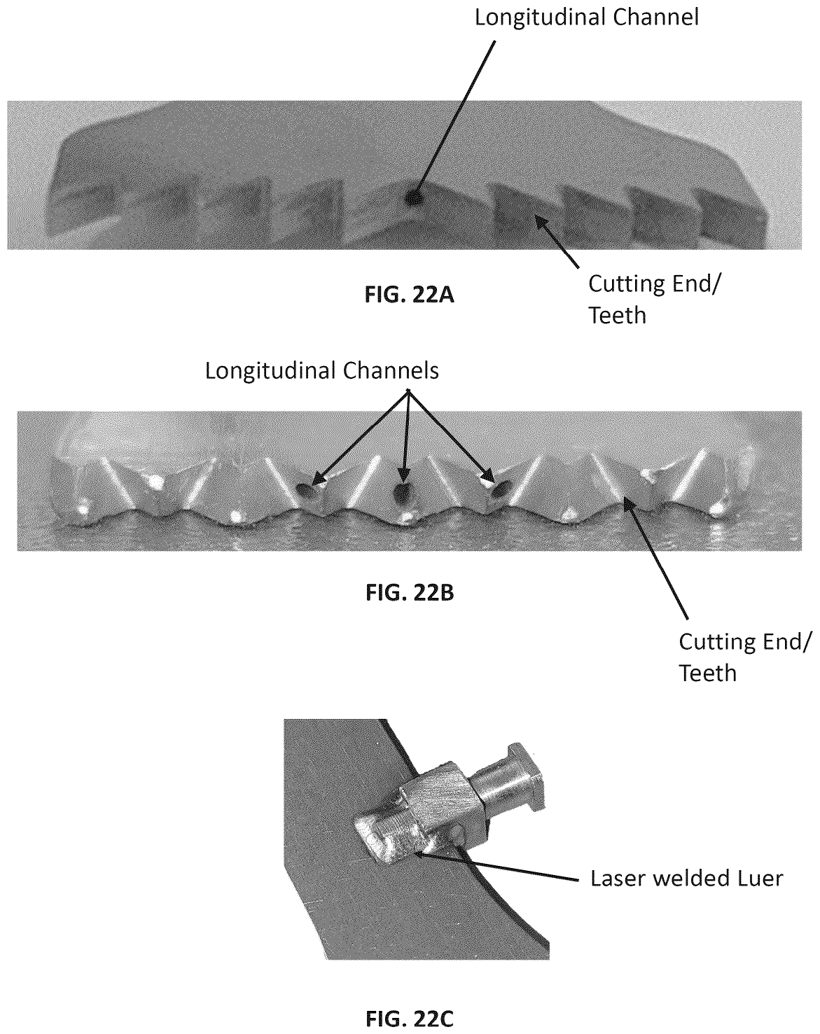

FIG. 22A shows an example saw blade with a 1.35 mm thick body and one (1) longitudinally-extending channel that is formed internal to the saw blade body. FIG. 22B shows an example saw blade with a 1.35 mm thick body and three (3) longitudinally-extending channels that are formed internal to the saw blade body. FIG. 22C shows an example inlet (e.g., Luer) that is laser welded onto a lateral surface over a saw blade body, so as to provide a fluid flow path through the inlet and into a lateral channel formed internal to a saw blade, such as is shown, for example, in FIGS. 17-21C.

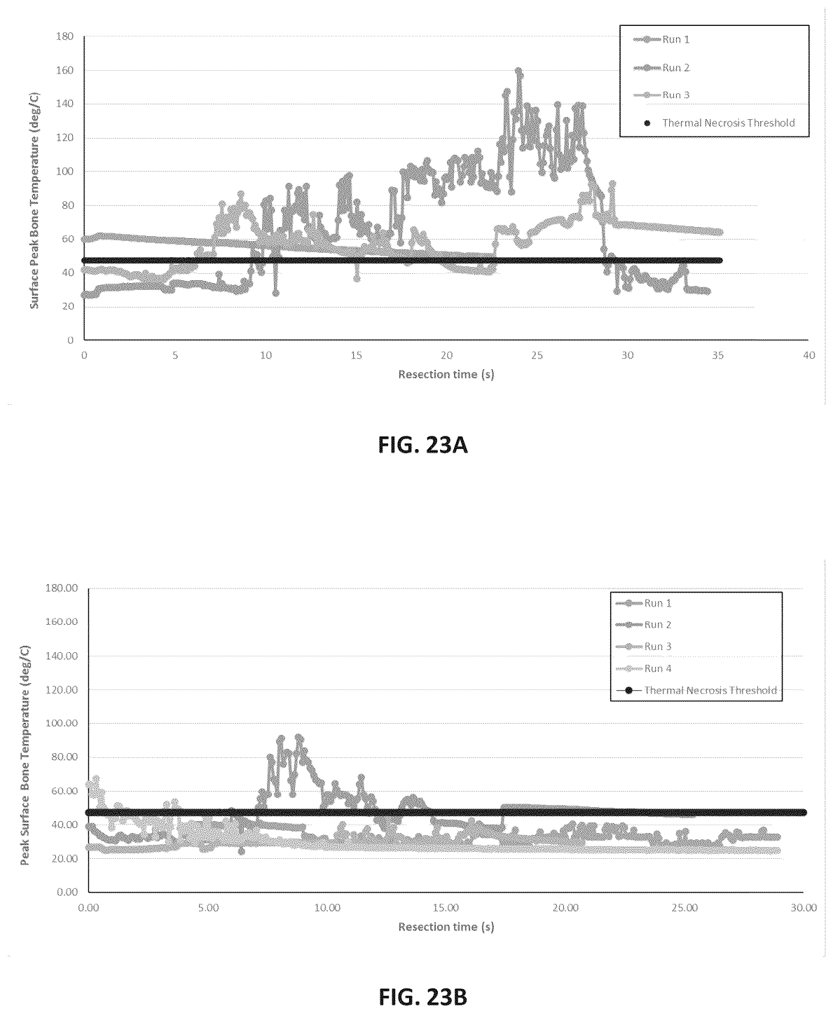

FIG. 23A is a temporal plot of bone surface temperature during a bone resection procedure using a conventional (i.e., non-irrigated) saw blade. FIG. 23B is a temporal plot of bone surface temperature during a bone resection procedure using an example internally water-cooled saw blade according to the examples disclosed herein. The temporal plots shown in FIGS. 23A and 23B were obtained using a FLIR thermal imaging camera (FLIR X8500sc). By comparing FIGS. 23A and 23B, it is apparent that the amount of time that the bone is above the minimum threshold temperature (i.e., 47° C.) for thermal necrosis is greater for the conventional saw blade compared to the internally water-cooled saw blade.

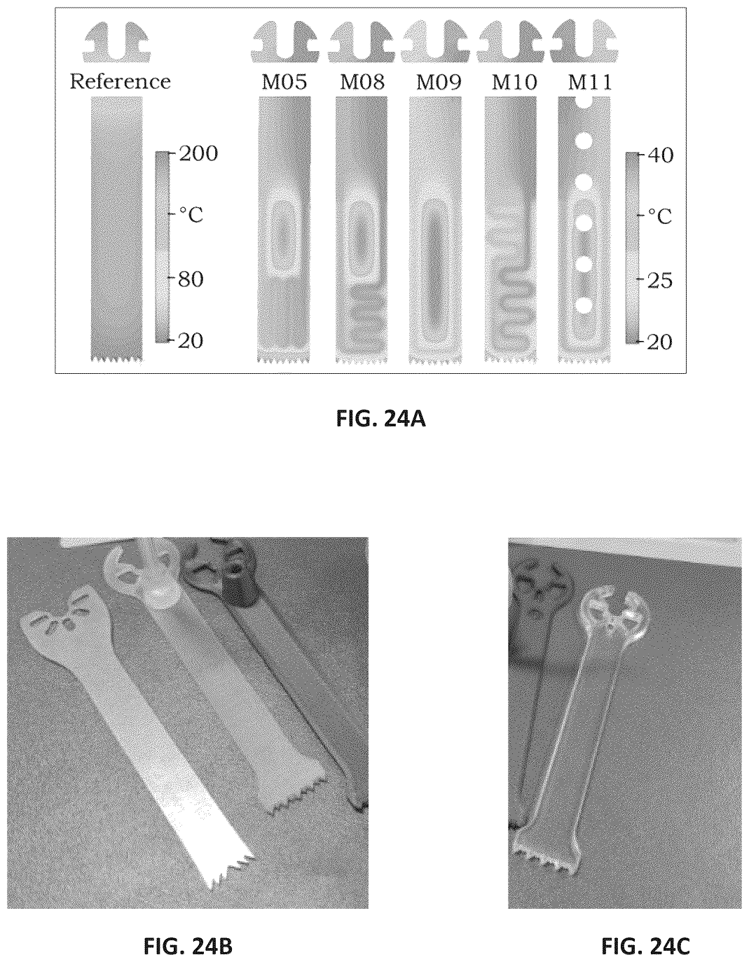

In an alternative example, the internal flow channels disclosed herein can be created using digital additive manufacturing, which provides greater freedom for designing the internal manifold. FIG. 24A is a temperature plot of a conventional saw blade (“Reference”) and also of different internally water-cooled saw blades of different sizes and flow channel designs. FIGS. 24B and 24C are photographs of internally water-cooled saw blades.

While the present disclosure refers to certain examples, numerous modifications, alterations, and changes to the described examples are possible without departing from the sphere and scope of the present disclosure. Accordingly, it is intended that the present disclosure not be limited to the described examples, but that it has the full scope defined by the language of the specification, and equivalents thereof, as would be understood by persons having ordinary skill in the art. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the descriptions of such examples herein are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

We claim:

1. A bone cutting device selected from one of a saw blade or a burr, comprising:
a proximal end configured to attached to a surgical instrument;
a distal end including a cutting end configured to cut bone; and
one or more internal channels formed in the cutting device, the one or more internal channels arranged and configured to receive a flow of a cooling medium through at least a portion of the cutting device, the cooling medium being supplied to the distal end.
